(19) United States
(12) Patent Application Publication
(10) Pub. No.: US 2006/0101537 A1
(43) Pub. Date: May 11, 2006

(54) Eucalyptus urophylla transformation and selection

(75) Inventors: Shujun Chang, N. Charleston, SC (US); Robert D. Thomas, Summerville, SC (US); Levis W. Handley, Takoma Park, MD (US); Marie B. Connett, Canberra (AU); Randy L. Hamilton, Charleston, SC (US)

Correspondence Address: FOLEY AND LARDNER LLP;SUITE 500, 3000 K STREET NW, WASHINGTON, DC, 20007, US

(73) Assignee: ArborGen, LLC

(21) Appl. No.: 11/158,342

(22) Filed: Jun. 22, 2005

Related U.S. Application Data
(63) Continuation-in-part of application No. 10/981,742, filed on Nov. 5, 2004.

(51) Int. Cl. A01H 1/00; C12N 15/82

(52) U.S. Cl. 800/278; 800/294

(57) ABSTRACT

The present invention relates to a method for transforming and selecting plant explants. The transformation method includes pre-culturing the explants in the presence of an Agrobacterium inducer and exposing the transformed explants to a shoot regeneration media that accelerates shoot development. Plants generated from this transformation method are provided. In particular, methods for obtaining transgenic E. grandis×E. urophylla cells and therefrom regenerating stably transformed E. grandis×E. urophylla trees are provided. The invention also provides media, methods, and plasmids for selecting and regenerating plants.

### CROSS REFERENCE TO RELATED APPLICATIONS

This application is a continuation-in-part of U.S. application Ser. No. 10/981,742, filed Nov. 5, 2004.

### FIELD OF THE INVENTION

The present invention relates to a genotype-independent method for transforming tree cells with foreign DNA and regenerating stably transformed trees therefrom. In particular, this invention teaches an Agrobacterium-mediated gene transfer method for transforming and regenerating transgenic plants from tree explants. The transformation process described herein utilizes a pre-culture medium that stimulates cell division and a shoot regeneration medium that accelerates shoot development. Plants generated from this transformation process are provided. In particular, the invention provides methods for increasing transformation efficiency and shoot regeneration from elite and recalcitrant clones. The invention also relates to media, methods, and plasmids for selecting and regenerating plants, particularly forest trees.

### BACKGROUND

Plant genetic engineering has provided great potential for the improvement of commercially important plant species. In recent years, the genetic engineering of trees has gained momentum and finds particular application in the pulping and timber industries. Several established tree transformation systems exist for such species as sweetgum (Sullivan and Lagrinini 1993), European larch (Huang et al. 1991), yellow poplar (Wilde et al. 1992) and many Populus sp. (Minocha et al. 1986, Fillatti et al. 1988, De Block 1990, Brasileiro et al. 1992, Tsai et al. 1994). Species in the Populus genera have served as the model systems for the genetic engineering of trees (Kim et al. 1997). Various traits, such as insect resistance and herbicide tolerance, have been engineered into these tree species (Klopfenstein et al. 1993, De Block 1990). Thus, the potential for genetically engineering tree species is great for commercially important tree species, including Eucalyptus.

Eucalyptus plants comprise more than five hundred species and hybrids. Eucalyptus has a high growth rate, adapts to wide range of environments, and displays little susceptibility to insect damage. In addition to its exceptional growth properties, Eucalyptus trees provide the largest source of fibers for the paper industry. Fibers from hardwood species, such as Eucalyptus, are generally much shorter than fibers from softwoods, such as pine. The shorter fibers produced from Eucalyptus results in the production of pulp and paper with desirable surface characteristics, including smoothness and brightness, but low tear or tensile strength. As a timber, Eucalyptus provides tall, straight timber with a medium to high density. Eucalyptus timber is general-purpose; finds use in the plywood and particleboard industry, furniture industry; and provides a source of firewood and construction materials.

Most reports demonstrating transformation of Eucalyptus use Eucalyptus seedlings rather than elite genotypes obtained through breeding programs. For example, WO 99/48355 describes a method for transforming young leaf explants from seedlings of E. grandis and E. camaldulensis. Even though the transformation was successful, there are two main problems with the described method. First, the regeneration protocol works for seedling explants, but it does not work with explants from elite genotypes. Second, even with seedling explants and the claimed improvements, the transformation efficiency is limited to 2.2% or lower for cotyledon explants of the two species and the hypocotyl explants of E. camaldulensis. An improved transformation and regeneration protocol was reported for E. camaldulensis seedlings by Ho et al., Plant Cell Reports 17:675-680 (1998); but, the protocol was not repeatable even with E. camaldulensis seedlings. Thousands of explants were cultured, and transgenic callus lines were produced, but the number of shoots recovered was minimal. Hartcourt et al. Molecular Breeding 6:306-315 (2000) reported the transformation of E. camaldulensis seedlings with the insecticidal cry3A gene and the herbicide resistant bar gene. Although five herbicide resistant callus lines were regenerated from an unnumbered explants derived from fifty seedlings, one line was difficult to propagate in culture or in the greenhouse, and another line was proven to be an escape. Moreover, the line that was difficult to propagate was one of the two lines that were analyzed at molecular level, and it was the only line with a single insertion. These studies indicate that even when the recovery of transgenic Eucalyptus plants is possible, the low transformation efficiency precludes delivery of a desired gene into many genotypes. Thus, a need continues to exist for a genotype-independent method of Eucalyptus transformation and regeneration.

Although micropropagation of Eucalyptus seedlings has been performed, de novo shoot regeneration has been limited to seedlings instead of the selected or “elite” clones of commercially important Eucalyptus species and hybrids. Elite genotypes, which arise through successive rounds of breeding, are valued for their combination of economically desirable traits. Unlike seedling transformation, which requires a large number of genotypes to ensure co-segregation of growth traits with the desired trait conferred by transgene expression, the transformation of elite clones would provide an efficient and advantageous system for genetically engineering tree species. Elite genotypes can be selected based on many years of clonal field test with a large number of starting genotypes. Like many other fast growing hardwood tree species, it takes years of field evaluation before relatively accurate predictions of a trait can be made for Eucalyptus. Therefore, if seedlings are used for genetic engineering, an even larger number of genotypes is needed for successful selection of a growth trait together with a desired trait conferred by transgene expression.

Both GB 2298205 (WO/9625504) and EP 1050209 claimed the use of 1-(2-chlorl-4-pyridyl)-3-phehylurea or N-(2-chloro-4-pyridyl)-N′-phenylurea (4-PU or 4CPPU) as the primary cytokinin for regeneration of transgenic shoots. In both cases, antibiotics were used as selection agents. WO/9625504 demonstrates the transformation of explants from mature genotypes, but the inventors used seedling explants for E. globules and E. nitens to demonstrate transformation. EP 1050209 uses a vertical rotary culture system for inducing formation of transgenic primordia. Although transformation was demonstrated with rejuvenated explants from mature trees, the transformation efficiency was calculated based on transgenic callus production and there was no indication of the frequency for transgenic plant production. Since efficient de novo shoot regeneration is critical for genetic engineering, there is a need to develop a highly efficient regeneration system for the selection of clones from commercially important Eucalyptus species and hybrids.

Accordingly, there is a need to increase the frequency of Eucalyptus cells and regenerate stably transformed plants from clones of recalcitrant species and hybrids.

Accordingly, there is a need to increase the transformation and regeneration efficiency of E. grandis×E. urophylla recalcitrant to transformation.

### SUMMARY OF THE INVENTION

Contemplated in the present invention is a method for transforming at least one cell of a E. grandis×E. urophylla explant with a foreign DNA, comprising (i) pre-culturing a tree explant on a medium comprising an inducer of Agrobacterium; (ii) exposing said explant to an Agrobacterium strain containing a transformation vector carrying said foreign DNA; (iii) selecting a transformed explant, wherein said foreign DNA is transferred to at least one cell of said transformed explant; and (iv) regenerating said transformed explant to produce a complete plant. Preferably, the inducer of Agrobacterium is acetosyringone and the concentration of said acetosyringone is from about 10 to about 400 mg/l. Also preferred, the concentration of said acetosyringone is from about 5 to about 200 mg/l and the medium further comprises auxin and/or cytokinin.

The invention contemplates a pre-culturing step comprising culturing a plant explant on a nutrient medium before Agrobacterium transformation. The pre-culture medium comprises an inducer of Agrobacterium, such as acetosyringone. In one embodiment, the explant is pre-cultured in the dark from about 1 to about 6 days. Preferably, the explant is pre-cultured for about 4 days. In one embodiment, the pre-culture medium may optionally comprise plant growth regulators, including auxin and cytokinin

In another embodiment, the auxin is selected from the group consisting of NAA, 2,4-D, IBA, and IAA. In one embodiment, the concentration range of any one of NAA, 2,4-D, IBA, and IAA is from about 0.1 to about 10 mg/l. Preferably, the concentration range is from about 0.2 to about 5 mg/l. More preferably, the concentration range is from about 0.2 to about 3 mg/l.

In another embodiment, the cytokinin is selected from the group consisting of zeatin, kinetin, and BA. In one embodiment, the concentration range of any one of said zeatin, kinetin, and BA is from about 0.25 to about 15 mg/l. Preferably, the concentration range is from about 1 to about 10 mg/l. More preferably, the concentration range is from about 1 to about 6 mg/l.

In another embodiment, the explant is at least one of a leaf, a petiole, an internodal tissue, floral tissue, embryogenic tissue, or embryogenic culture.

In one embodiment, the tissues are selected independent of age or developmental stage.

In another embodiment, the method is genotype-independent.

In another embodiment, all of the cells of said transformed explant comprise the foreign DNA.

The invention also contemplates a method for producing a non-chimaeric E. grandis×E. urophylla tree, comprising (i) pre-culturing a E. grandis×E. urophylla explant on a medium comprising an inducer of Agrobacterium; (ii) exposing said explant to an Agrobacterium strain harboring a vector capable of transferring a gene to a plant cell; (iii) selecting a transformed explant; and (iv) growing said explant to produce a non-chimaeric tree.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 provides a schematic representation of the vector pWVC20.

FIG. 2 provides a schematic representation of the vector pWVC21.

FIG. 3 provides a schematic representation of the vector pWVC23.

FIG. 4 provides a schematic representation of the vector pWVC24.

FIG. 5a provides a schematic representation of the vector pWVC25.

FIG. 5b provides a schematic representation of the vector pWVC26.

FIG. 6 provides a schematic representation of the vector pWVC30.

FIG. 7 provides a schematic representation of the vector pWVC33.

FIG. 8a provides a schematic representation of the vector pWVC34.

FIG. 8b provides a schematic representation of the vector pWVC35.

FIG. 9 provides a schematic representation of the vector pWVK192.

FIG. 10 provides a schematic representation of the vector pARB 1001.

FIG. 11 provides a schematic representation of the vector pWVR133.

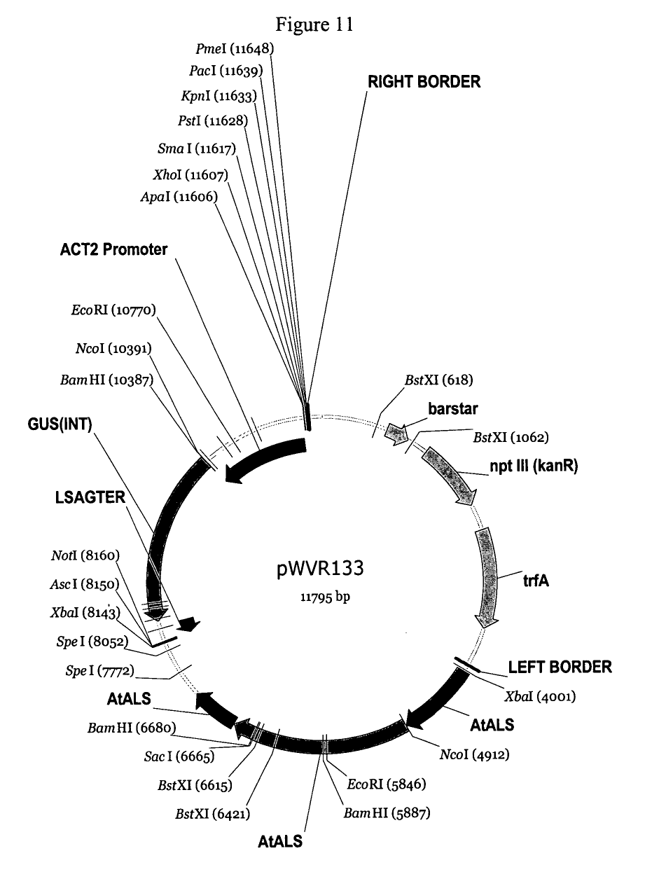

FIG. 12 provides a schematic representation of the vector pWVR31.

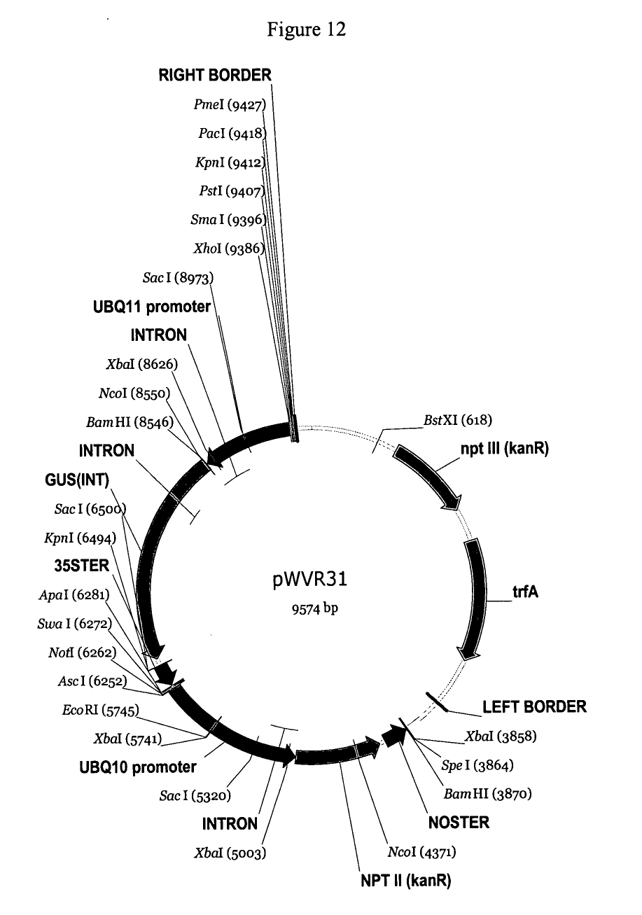

### DETAILED DESCRIPTION OF THE INVENTION

The methods of the present invention for genotype-independent tree transformation overcome the low transformation efficiencies obtained from the transformation of elite genotypes. In its broadest aspect, the methods relate to increasing the efficiencies of transformation and shoot regeneration from tree explants of recalcitrant species and hybrids. In one aspect, the methods provide increase in transformation and regeneration efficiencies of recalcitrant species and hybrids, such as E. grandis×E. urophylla. Increases in transformation efficiency are accomplished by pre-culturing the explants on a medium that stimulates cell division. The shoot regeneration frequency of the transformed explants is improved by culturing the explants on a medium that promotes shoot regeneration.

In the description that follows, a number of scientific and technical terms are used extensively. Unless defined otherwise, all technical and scientific terms used herein share the same meaning as commonly understood by one of ordinary skill in the art to which this invention belongs. The following definitions are provided to facilitate understanding of the invention.

Abaxial, as used herein, refers to the lower side of a leaf.

Agrobacterium inducer refers to a molecule that induces expression of Agrobacterium virulence genes that code for products that control excision and delivery of the T-DNA into the host plant nucleus. In the present description, Agrobacterium inducers were added to both the pre-culture medium and the Agrobacterium culture medium. In this invention, Agrobacterium inducers include, but are not limited to, phenolic compounds, such as acetosyringone. Addition of an inducer improves Agrobacterium infection frequency and consistency.

Agrobacterium-mediated transformation is a method by which DNA is stably inserted into the genome of a plant cell through the use of the Ti (tumor-inducing) plasmid from Agrobacterium tumefaciens. A small portion of the Ti plasmid, known as the T-DNA, is incorporated into the nucleus of the host plant cell. Alternatively, the R1 plasmid from Agrobacterium rhizogenes may be used for transformation. In Agrobacterium-mediated transformation, the gene(s) or DNA intended for plant introduction is positioned between the left and right borders of the T-DNA.

Antioxidant, as used herein, refers to a compound that minimizes the exudation of phenolic materials from a plant explant. In the present invention, ascorbic acid may be used as an antioxidant.

In the present description, the term auxin encompasses a class of plant growth regulators that are characterized principally by their capacity to stimulate cell division in excised plant tissues. In addition to their role in cell division and cell elongation, auxins influence other developmental processes, including root initiation. In the present invention, auxin and auxin-type growth regulators include, but are not limited to, naphthaleneacetic acid (NAA), 2,4-Dichlorophenoxy acetic acid (2,4-D), indole-3-butyric acid (IBA), and indole-3-acetic acid (IAA).

Casein hydrolysate-like compound refers to a composition that is structurally and functionally related to casein hydrolysate. In the context of the phrase “casein hydrolysate-like compound, the term denotes a compound that has a similar composition of amino acids as casein hydrolysate, and achieves the same function as casein hydrolysate, but differs in one or more components. For example, a casein hydrolysate-like compound can possess elevated concentrations of amino acids such as glutamine and arginine, important amino acids for growing and maintaining regenerable cells of certain plants in plant tissue culture.

A cloning vector is a genetic element, such as a plasmid, cosmid, or bacteriophage, that has the capability to replicate autonomously in a host cell. Cloning vectors comprise one or a small number of restriction endonuclease recognition sites in which foreign DNA sequences can be inserted in a determinable orientation and a marker gene that encodes a product suitable for the identification and selection of cells transformed with the cloning vector. Marker genes include genes whose products confer antibiotic or herbicide resistance. The insertion of a foreign DNA sequence into a cloning vector does not interfere with an essential biological function of the cloning vector or the marker gene.

Cytokinin refers to a class of plant growth regulators that are characterized by their ability to stimulate cell division and shoot organogenesis in tissue culture. In the present invention, cytokinins include, but are not limited to, N⁶-benzylaminopurine (BAP), N⁶-benzyladenine (BA), zeatin, kinetin, thiadiazuron (TDZ), 2-isopentenyladenine (2ip), and 4-CPPU (N-(2-chloro-4-pyridyl)-N′-phenylurea)).

Derivative refers to a substance that is structurally and functionally related to another substance. In the context of the phrase “derivative of casein hydrolysate,” the term denotes a substance that has a similar composition of amino acids as casein hydrolysate, and achieves the same function as casein hydrolysate, but differs in one or more components. For example, a derivative of casein hydrolysate may have more arginine and/or less valine than a typical casein hydrolysate.

As used herein, elite genotype refers to commercially important genotypes obtained and selected for through successive breeding programs.

The term explant refers to plant tissue that is a target for transformation. Preferred explants comprise leaf, petiole, floral tissue, internodal tissues, and embryogenic tissues harvested from plants grown in vivo and/or in vitro.

The term expression refers to the biosynthesis of a gene product. For example, expression of a gene involves transcription of the DNA sequence into mRNA and translation of the mRNA into one or more polypeptides. The RNA generated may code for a protein or polypeptide or may code for an RNA interfering, or antisense molecule.

An expression vector is a genetic element comprising a gene sequence that is expressed in a host cell. Typically, the expression of the gene sequence is controlled by several regulatory elements, including constitutive and inducible promoters, tissue-preferred regulatory elements, and enhancers. Such a gene is said to be “operably linked” to the regulatory elements.

A foreign DNA is DNA isolated from another species or from the species of interest and re-introduced into the same species. The DNA may be a structural gene, an antisense gene, DNA fragments, etc.

A gene is a heritable DNA sequence that is transcribed into messenger RNA (mRNA) which is then translated into a sequence of amino acids characteristic of a polypeptide.

A non-chimaeric transgenic plant is the product of a transformation event wherein essentially all of the cells are transformed and a foreign DNA is transferred to progeny.

Operably linked describes combining two or more molecules in such a fashion that in combination they function properly in a plant cell. For instance, a promoter is operably linked to a structural gene when the promoter controls transcription of the structural gene.

As used herein, plant is any of various photosynthetic, eukaryotic, multicellular organisms of the kingdom Plantae characteristically producing embryos, containing chloroplasts, and having cellulose cell walls. As part of a plant, a plant tissue may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as turfgrass, wheat, maize, rice, barley, oat, sugar beet, potato, tomato, tobacco, alfalfa, lettuce, carrot, strawberry, cassaya, sweet potato, geranium, soybean, oak, pine, fir, acacia, eucalyptus, walnut, and palm. According to the present invention plant tissue also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are conifers such as pine, fir and spruce, monocots such as Kentucky bluegrass, creeping bentgrass, maize, and wheat, and dicots such as Eucalyptus, Acacia, aspen, Sweetgum, poplar, cotton, tomato, lettuce, Arabidopsis, tobacco, and geranium.

Plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as leaves, roots, or seeds. Such promoters are referred to as tissue-preferred promoters. Promoters which initiate transcription only in certain tissue are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Pre-culture medium, as used herein, is the nutrient medium upon which plant explants are cultured prior to transformation with Agrobacterium and is needed for increasing transformation efficiency and plant regeneration. The pre-culture medium comprises an inducer of Agrobacterium, such as acetosyringone. The pre-culture medium comprises plant growth regulators, including auxin and cytokinin, especially an elevated level of auxin to stimulate the cell division and growth.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA construct, which also is inherited by the progeny plant from its parent. The term “progeny” as used herein, also may be considered to be the offspring or descendants of a group of plants.

Shoot regeneration medium is the inventive medium designed to regenerate transgenic shoots. The shoot regeneration medium comprises inorganic salts, a mixture of amino acids and vitamins, an antioxidant, organic nitrogen, and plant growth regulators.

Somatic embryogenesis is a method of clonal propagation wherein the embryo develops from vegetative or somatic tissue rather than as a product of gametic fusion.

Stably transformed refers to a transgenic plant that is capable of transmitting foreign DNA to progeny.

Structural gene is a DNA sequence that is transcribed into messenger RNA (mRNA) which is then translated into a sequence of amino acids characteristic of a specific polypeptide.

Substantially free refers to the relative absence of a first compound from a second compound. The term denotes that less than 10%, 5%, 4%, 3%, 2%, 1% or even 0% of a first compound can be detected in a second compound.

A transgenic plant is a plant comprising foreign DNA. In this invention, a transgenic plant is derived from Agrobacterium-mediated transformation. Preferably, the transgenic plant is fertile and capable of transmitting the foreign DNA to progeny plant through sexual reproduction.

Transcription and translation terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product.

Tree, as used herein, refers to any perennial vegetation that accumulates a wood core. Trees include angiosperms and gymnosperm species and hybrids. Examples of trees include poplar, Eucalyptus, Douglas fir, pine, sugar and Monterey, nut trees, e.g., walnut and almond, fruit trees, e.g., apple, plum, citrus and apricot, and hardwood trees, such as ash, birch, oak, and teak. Of particular interest are conifers such as pine, fir, spruce, Eucalyptus, Acacia, aspen, Sweetgum, and poplar.

The present invention provides genotype-independent methods for transforming tree explants and generating transgenic progeny therefrom. The methods of the present invention contemplate pre-culturing tree explants in the presence of an Agrobacterium inducer. The methods of the present invention further envision culturing the transformed explants on a shoot regeneration medium comprising amino acids, vitamins, plant growth regulators, glucose, and an antioxidant.

The methods of the instant invention provide a genotype-independent method of Eucalyptus explant transformation and shoot regeneration. Any Eucalyptus explant may be transformed by the methods of the instant invention, including Eucalyptus trees grown in natural environments and Eucalyptus explants clonally propagated. The explant may be selected from any Eucalyptus species or hybrid, including Eucalyptus alba, Eucalyptus bancroftii, Eucalyptus botryoides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dalrympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus grandis, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-angelica, Eucalyptus obliqua, Eucalyptus occidentalis, Eucalyptus obtusiflora, Eucalyptus oreades, Eucalyptus pauciflora, Eucalyptus polybractea, Eucalyptus regnans, Eucalyptus resinifera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo, and Eucalyptus youmanni.

In particular, the transgenic plant may be Eucalyptus grandis or its hybrids.

The methods of the instant invention contemplate the transformation of Eucalyptus explants obtained from a stock culture of elite Eucalyptus genotypes. Micropropagated shoot cultures may be generated by harvesting newly flushed apical or axillary shoots and surface sterilizing the tissues in a sterilization solution. Sterilization solutions, such as 1-5% bleach solution, are known in the art and repeated rinsing with sterile, distilled water can be performed. Eucalyptus stock cultures can be maintained as shoot clusters on a maintenance medium comprising inorganic salts, carbon sources, vitamins, and cytokinins. In the present invention, stock cultures are maintained on Eucalyptus Maintenance (EM) medium (Table 1) comprising of modified Woody Plant Medium (WPM) salts (Loyd and McCown, 1980) and N⁶-benzyladenine (BA). Alternatively, other salt media, such as MS medium (Murashige and Skoog 1962) or Lepoivre medium, may be used.

TABLE 1
Eucalyptus Maintenance Medium (EM medium)

| Medium | Amount per Liter | |
|---|---|---|
| Components | of Medium | |
| WPM salts | 1 | package (Sigma) |
| Ca(NO₃)₂.4H₂O | 3.7 | g |
| MgSO₄.4H₂O | 0.37 | g |
| Nicotinic Acid | 0.5 | mg |
| Thiamine.HCl | 0.5 | mg |
| Pyridoxin.HCl | 0.5 | mg |
| D-Pantothenic Acid | 1.0 | mg |
| Myo-inositol | 0.1 | g |
| BA | 0.1-1 | mg |
| Bacto-agar | 5-8 | g |

The present invention provides a method of genotype-independent transformation. The methods of the present invention teach the transformation of explants independent of age and developmental stage. Tree explants obtained from the stock culture can be used for transformation. Tree explants may be selected from one of more of leaf, petiole, internodal, and floral tissues. In the present invention, leaf explants are selected due to their abundant supply and ease of transformation. The tip portion of leaves may be removed or punctured with a forcep or sharp scissors to increase the number of wounded cells. In the instant application, leaf explants are placed on the pre-culture medium abaxial side down.

In the instant application, plant explants are cultured on a pre-culture medium. A pre-culture medium is a nutrient medium upon which plant explants are cultured before Agrobacterium transformation. Specifically, the inventive pre-culture medium increases transformation efficiency and plant regeneration. The pre-culture medium comprises an inducer of Agrobacterium, such as acetosyringone. Alternatively, other Agrobacterium inducers may be used, such as hydroxyphenylpropanoids, phenolic compounds, and coniferin. Shoot regeneration medium with the modification of plant growth regulators and the organic composition was used in the present invention; however, other salt media, such as MS medium (Murashige and Skoog 1962), Woody Plant Medium (WPM) salts (Loyd and McCown, 1980) or Lepoivre medium, may be used. Optionally, the pre-culture medium may comprise the same levels of auxin and cytokinin or the organic composition of the shoot regeneration medium (Table 3). In the present invention, plant explants were pre-cultured for four days in the dark on the Pre-Culture Medium of Table 2. Other pre-culture media and time periods of culture may be used.

TABLE 2
Plant Pre-Culture Medium

| Components for 1 Liter of | |
|---|---|
| Medium | Grams (g) |
| KNO3 | 1 |
| NH4H2PO4 | 0.25 |
| MgSO4.7H2O | 0.25 |
| CaCl2.2H2O | 0.10 |
| FeSO4.7H2O | 0.0139 |
| Na2EDTA.2H2O | 0.01865 |
| MES (Duchefa m1501) | 600.0 |
| MS Micro (½ strength) | |
| MnSO4.H2O | 0.00845 |
| ZnSO4.7H2O | 0.0043 |
| CuSO4.5H2O | 0.0000125 |
| CoCl2.6H2O | 0.0000125 |
| KI | 0.000415 |
| H3BO3 | 0.0031 |
| Na2MoO4.2H2O | 0.000125 |
| Plant Growth | |
| Regulators | |
| Zeatin | 0.001-0.006 |
| IAA (Indo-3-acetic acid) | 0.0001-0.01 |
| Sugars | |
| Glucose/Sucrose | 20.0 |
| Myo-inositol | 0.100 |
| Amino acid and vitamin | |
| mix | |
| Nicotinic Acid | 0.010 |
| Thiamine | 0.010 |
| Ca Pantothenate | 0.001 |
| Pyridoxine | 0.001 |
| Biotin | 0.00001 |
| Ascorbic Acid | 0.050 |
| L-glutamine | 0.1 |
| Casein hydrolysate | 0.5 |
| Agrobacterium inducer | |
| Acetosyringone | 5-200 mg |
| Gelling Agent | |
| Gelrite | 1-6 |

The methods of the present invention teach pre-culturing the explants on a pre-culture medium containing a high concentration of auxin or auxin-type growth regulators. Preferably, the auxin or an auxin-type growth regulator is selected from the group consisting of naphthaleneacetic acid (NAA), 2,4-Dichlorophenoxyacetic acid (2,4-D), indole-3-butyric acid (IBA), and indole-3-acetic acid (LAA). More preferably the auxin is IAA. The concentration range of said auxin is from about 0.1 to about 10 mg/l. The preferred auxin concentration is from about 0.2 to about 5 mg/l. More preferably, the auxin concentration is from about 0.2 to about 3 mg/l.

Preferably, the methods of the present invention provide for pre-culturing tree explants on a pre-culture medium comprising sufficient cytokinin. The cytokinin is selected from the group consisting of N⁶-benzylaminopurine (BAP), N⁶-benzyladenine (BA), zeatin, kinetin, 4-CPPU (N-(2-chloro-4-pyridyl)-N′-phenylurea)), thiadiazuron (TDZ), and 2-isopentenyladenine (2ip). The concentration range of said cytokinin is from about 0.25 to about 15 mg/l. Preferably, the cytokinin concentration range is from about 1 to about 10 mg/l. More preferably, the cytokinin concentration range is from about 1 to about 6 mg/l.

The methods of the present invention provide for pre-culturing tree explants on a pre-culture medium comprising an Agrobacterium inducer. The inducer may be acetosyringone. The concentration range of acetosyringone is from about 10 to about 400 mg/l. Preferably, the concentration range is from about 5 to about 200 mg/l.

The present invention contemplates the method of optionally pre-culturing tree explants on a pre-culture medium comprising both an auxin and an Agrobacterium inducer. The invention demonstrates that the combination of an auxin with an Agrobacterium inducer results in higher infection rates than either component independently. The explants can be pre-cultured on said pre-culture medium for about 1 to about 6 days prior to the introduction of Agrobacterium. Preferably, the explants are pre-cultured for about 4 days. The pre-culturing step can occur under both dark and light conditions; however, it is preferable to culture in the dark.

Eucalyptus explant transformation is performed with different strains of A. tumefaciens harboring a transformation vector. One such vector is GV2260, comprising a GUS gene operably linked to a promoter, such as an actin promoter or a constitutive promoter. The vector will also carry a herbicide resistance gene operably linked to a promoter. In the present invention, the acetolactate synthase (ALS) gene confers herbicide resistance and is driven by its Arabidopsis native promoter. See U.S. Pat. No. 6,225,105. An A. tumefaciens culture suspended in induction medium (AIM formulation) is dripped by pipette on to the explants such that all cut edges are exposed to the bacteria. Alternatively, the explants may be transformed by vacuum infiltration, floral dip, and other methods of Agrobacterium-mediated transformation that are well known in the art. For a review of Agrobacterium-mediated transformation, see Gelvin, S B. Microbiol. Mol Biol Rev 67:1:16-37 (2003). Upon introduction of Agrobacterium, the explants are co-cultivated with the Agrobacterium for about 3 days on the same medium. Following co-cultivation, the explants are transferred to a shoot regeneration medium for the recovery of transgenic shoots.

The present invention teaches methods for shoot regeneration wherein transformed explants are cultured on a medium comprising a mixture of amino acids and vitamins, plant growth regulators, glucose, and an antioxidant. One such complete medium formulation is listed in Table 3 and is called Euc Regeneration medium. An antioxidant, such as ascorbic acid, may be added to the Euc Regeneration medium to minimize the exudation of plant phenolic compounds. Glucose and glutamine may be added to the medium to accelerate shoot regeneration. Antibiotics, such as carbenicillin, cefotaxime, and timentin can also be included in the medium to prevent bacterial overgrowth. Timentin is the preferred antibiotic. The antibiotic concentration ranges from about 75 to 800 mg/l. Preferably, the antibiotic concentration is about 400 mg/l.

The Euc Regeneration medium contains a mixture of amino acids that do not interfere with amino acid biosynthesis. In the present invention, the Euc Regeneration medium does not interfere with sulfonylurea-based herbicide selection. Preferably, the Euc Regeneration medium does not have branched chain amino acids (e.g. leucine, isoleucine, and valine). More preferably, the media has an amino acid mixture that replaces casein hydrolysate. Most preferably, the amino acid mixture has amino acids important for plant tissue culture growth.

TABLE 3
Eucalyptus Regeneration Medium

| Components for 1 Liter of | |
|---|---|
| Medium | Grams |
| KNO₃ | 1 |
| NH₄H₂PO₄ | 0.25 |
| MgSO₄.7H₂O | 0.25 |
| CaCl₂.2H₂O | 0.10 |
| FeSO₄.7H₂O | 0.0139 |
| Na₂EDTA.2H₂O | 0.01865 |
| MES (Duchefa m1501) | 600.0 |
| MS Micro (½ strength) | |
| MnSO₄.H₂O | 0.00845 |
| ZnSO₄.7H₂O | 0.0043 |
| CuSO₄.5H₂O | 0.0000125 |
| CoCl₂.6H₂O | 0.0000125 |
| KI | 0.000415 |
| H₃BO₃ | 0.0031 |
| Na₂MoO₄.2H₂O | 0.000125 |
| Plant Growth Regulators | |
| Zeatin | |
| NAA (naphthalene acetic acid) | |
| Sugars | |
| Glucose/Sucrose | 20.0 |
| Myo-inositol | 0.100 |
| Amino acid and vitamin mix | |
| Nicotinic Acid | 0.010 |
| Thiamine | 0.010 |
| Ca Pantothenate | 0.001 |
| Pyridoxine | 0.001 |
| Biotin | 0.00001 |
| Ascorbic Acid | 0.050 |
| L-glutamine | 0.1 |
| Arginine | 0.0258 |
| Glycine | 0.00199 |
| Lysine | 0.0508 |
| Methionine | 0.0132 |
| Phenylalanine | 0.0257 |
| Serine | 0.00904 |
| Threonine | 0.00852 |
| Tryptophan | 0.0122 |
| Tyrosine | 0.0127 |
| Gelling Agent | |
| Gelrite | 3.0 |

In the present invention, there is a 4-day recovery period before the selection of transformed explants. To select for transformed explants, any selectable marker is added to the regeneration medium. Selectable markers include herbicides and antibiotics. Additionally, any screenable marker may be used to select a transformed plant. Examples of screenable markers include B-glucuronidase (GUS), green fluorescent protein (GFP), and luciferase. In the present invention, an herbicide selection agent is used. While the herbicide of the present invention is Ally, any herbicide may be used. Other herbicides include Oust and Liberty. The herbicide concentration may vary depending on the sensitivity of the explants from a specific species. The selected explants are subcultured every two to three weeks until the formation of adventitious buds. Transformed adventitious shoots are separated from shoot clumps and are stained for GUS (B-glucuronidase) expression. Jefferson et al. EMBO:6:13:901-3907 (1987). A reporter gene assay, such as GUS, is used to determine transformation efficiency and to ensure that the transformed shoots are not escapes or chimeras.

Upon confirmation of transformation via expression of a screenable marker gene, Southern blot analysis, PCR analysis, or other method known in the art, the transformed shoots are preferably transferred to a medium for shoot elongation. The present invention contemplates a shoot elongation medium comprising MS salts, sucrose, auxin, and giberellic acid. NAA is the preferred auxin and GA3 is the preferred giberellic acid. The shoots are cultured on the shoot elongation medium for about 10 to about 14 days, preferably under dark conditions. For the elongation of E. dunii clones, additional auxin needs to be added to the elongation media and the shoots should be cultured in the dark for the duration of the elongation.

Following shoot elongation, the shoots are excised and transferred to a root induction medium. Depending on the light conditions, it may be necessary to add plant growth regulators, such as auxins, to the root induction medium. The methods of the present invention teach shoot excision at the node or immediately below the node. More preferably, the shoots are excised from a node near the shoot apex. One such rooting medium (Table 4) is comprised of BTM-1 nutrients (Chalupa 1988), activated carbon (MeadWestvaco, Nuchar), and an extra amount of CaCl₂. Alternatively, other low-salt media may be used for the root induction medium, such as Woody Plant Medium and ½ strength MS.

Depending on the light conditions, the rooting media may contain growth regulators such as auxins to induce root formation. For example, under dark conditions that induce etiolation and auxin production in the shoot apical meristem, it may not be necessary to include auxin in the rooting medium. Additionally, if the shoots are excised in the dark, it may not be necessary to limit the origin of excision to the nodal regions and/or the shoot apex. Alternatively, if the rooting step occurs under light, it may be necessary to include auxin in the rooting medium. Furthermore, if the rooting step occurs in the light, it is preferable to excise the shoots at a node near the shoot apical meristem.

TABLE 4
Preferred rooting medium for Eucalyptus

| BTM-1 Media Components | mg/L |
|---|---|
| NH₄NO₃ | 412 |
| KNO₃ | 475 |
| Ca(NO₃)₂.4H₂O | 640 |
| CaCl₂.2H₂O | 440* |
| MgSO₄.7H₂O | 370 |
| KH₂PO₄ | 170 |
| MnSO₄.H₂O | 2.3 |
| ZnSO₄.7H₂O | 8.6 |
| CuSO₄.5H₂O | 0.25 |
| CoCl₂.6H₂O | 0.02 |
| KI | 0.15 |
| H₃BO₃ | 6.2 |
| Na₂MoO₄.2H₂O | 0.25 |
| FeSO₄.7H₂O | 27.8 |
| Na₂EDTA.2H₂O | 37.3 |
| Myo-inositol | 100 |
| Nicotinic acid | 0.5 |
| Pyridoxine HCl | 0.5 |
| Thiamine HCl | 1 |
| Glycine | 2 |
| Sucrose | 20000 |
| Activated Carbon | 5000 |

*Additional 400 mg/l CaCl₂.2H₂O is added to the medium

For species and hybrids that are difficult to root, excised shoots can be pulse-treated with a medium having low levels of auxin to induce shoot formation. For example, a medium comprising 0.25 mg/l IBA may be used for pulse treatment. Preferably, the shoots are pulse-treated for about 5-14 days before transferring to BTM-1 medium having activated carbon.

The transformation method of the present invention can be used for introducing any foreign DNA into a Eucalyptus species or hybrid. Using the methods of the instant invention, any foreign DNA can be stably integrated into a plant cell and transmitted to progeny. For example, a gene involved in lignin biosynthesis, floral development, cellulose synthesis, nutrient uptake and transport, disease resistance, or enhanced resistance to an environmental condition can be introduced into a plant cell by the instant methods.

The methods of the present invention can be used for reducing gene expression in a Eucalyptus species or hybrid. Reduction of gene expression can be obtained by methods known in the art, including antisense suppression, co-suppression (sense suppression), and double stranded RNA interference. For a general review of gene suppression techniques, see Science, 288:1370-1372 (2000). Exemplary gene silencing methods are also provided in WO 99/49029 and WO 99/53050.

For antisense suppression, a cDNA sequence is arranged in a reverse orientation relative to the promoter sequence in a DNA construct. The cDNA sequence need not be full length relative to either the primary transcription product or fully processed mRNA. Generally, higher identity can be used to compensate for the use of a shorter sequence. Furthermore, the introduced sequence need not have the same intron or exon pattern, and identity of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3500 nucleotides is especially preferred. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other genes within a family of genes exhibiting identity or substantial identity to the target gene.

Another well known method of gene suppression in plants in sense co-suppression. Introduction of a nucleic acid sequence configure in the sense orientation provides an effective means by which to block the transcription of target genes. See, Assaad et al. Plant Mol. Bio. 22: 1067-1085 (1993); Flavell Proc. Natl. Acad. Sci. USA 91: 3490-3496 (1994); Stam et al. Annals Bot. 79: 3-12 (1997); Napoli et al., The Plant Cell 2:279-289 (1990); and U.S. Pat. Nos. 5,034,323, 5,231,020, and 5,283,184. For co-suppression, the introduced sequence need not be full length, relative to either the primary transcription product or fully processed mRNA. Preferably, the introduced sequence is not full length, so as to avoid a concurrent sense overexpression phenotype. In general, a higher identity in a shorter than full length sequence compensates for a longer, less identical sequence.

Using the methods of the instant invention, antisense suppression of a gene involved in lignin biosynthesis can be used to modify lignin content and/or composition in a transformed Eucalyptus plant. It has been demonstrated that antisense expression of sequences encoding cinnamyl alcohol dehydrogenase (CAD) in poplar, N. tabacum, and pine leads to the production of lignin having a modified monomeric composition. Grand et al., Planta 163: 232-37 (1985), Yahiaoui et al., Phytochemistry 49: 295-306 (1998), and Baucher et al., Plant Physiol. 112: 1479 (1996), respectively. Accordingly, the methods of the present invention can be used to stably transform and regenerate Eucalyptus species or hybrids having antisense suppression of a foreign DNA involved in lignin biosynthesis.

The methods of the present invention can be used for regulating floral development in a Eucalyptus species or hybrid. Several gene products have been identified as critical components for anther development and pollen formation. For example, premature degradation of callose, which is essential for the formation of the microspore cell wall and microspore release, is sufficient to cause male sterility. Worrall et al., Plant Cell 4:7:759-71(1992). Accordingly, several methods have been developed for producing male sterile plants. U.S. Pat. No. 5,962,769, for example, describes transgenic plants rendered male sterile via expression of avidin. Avidin can be expressed constitutively, in a non-tissue specific manner, or in anther-specific tissues. In addition, male sterility can be induced by antisense suppression of chalcone synthase A gene. van der Meer et al., The Plant Cell 4:253 (1992). By the methods of the present invention, male sterile Eucalyptus species or hybrid can be produced and regenerated.

The present invention provides plant media comprising a sulfonylurea or related herbicide and a derivative of casein hydrolysate, wherein the derivative is substantially free of branched chain amino acids. Casein hydrolysate, also known as casein powder or casamino acids, is a mixture of amino acids and short peptide fragments obtained from the hydrolysis of casein. While various amino acid compositions are reported in the literature, and various amino acid compositions may be obtained from various sources of casein and various means of hydrolysis, casein hydrolysate obtained from any source by any means normally comprises branched chain amino acids including valine, leucine and isoleucine, aromatic amino acids such as tryptophan, and other amino acids such as glycine. Additionally, while the composition varies, practitioners agree that the compound is critical for initiating growth of plant cells, particularly many types of embryogenic cultures and conifer plant cells, in vitro. Supplementation of the inorganic media components with reduced nitrogen components such as amino acids, and in particular casein hydrolysate, is viewed by those skilled in the art of woody plant tissue culture, and particularly conifer tissue culture, as an essential component to induce or maintain the growth of cultures that can subsequently be regenerated into plants. For example, the initiation, induction and/or maintenance media of embryogenic cell culture of conifer species routinely comprise casein hydrolysate. See e.g. U.S. Pat. Nos. 5,034,326, 5,036,007, 5,041,382, 5,236,841, 5,310,672, 5,491,090, 5,413,930, 5,506,136, 5,534,433, 5,534,434, 5,563,061, 5,610,051, 5,821,126, 5,850,032, 6,200,809 and 6,518,485.

Thus, regardless of whether the media of subsequent culturing steps comprises casein hydrolysate, small amounts of the compound persist. Such trace amounts can wreak havoc during selection. For example, minute quantities of casein hydrolysate can inhibit the effectiveness of selection agents such as sulfonylureas, imidazolinones and triazolopyrimidines, which work to identify transgenic plant cells following transfection by a vector of interest.

As noted above, these selection agents target acetolactate synthase (ALS), which catalyzes the first common step in a plant's biosynthetic pathway of the branched-chain amino acids valine, leucine and isoleucine. Mutant forms of ALS which are resistant to these agents are utilized as selectable markers in recombinant DNA constructs and are paired with selection agents to identify transformed plant cells.

Therefore, minute quantities of casein hydrolysate in the selection medium enable untransformed cells to grow in the presence of sulfonylureas, imidazolinones or triazolopyrimidines. These false positives impede the production of transgenic plants since more samples have to be evaluated in order to identify desired transformants.

The present invention eliminates this problem by modifying the composition of casein hydrolysate so as to be substantially free of branched-chain amino acids. In another embodiment, the derivative of casein hydrolysate comprises a higher percentage of amino acids that are important in plant tissue culture growth, such as arginine or glutamine.

In another aspect, the invention provides a media composition for selecting transgenic plants comprising a tryptophan analog, wherein the media is substantially free of tryptophan.

Feedback-insensitive forms of the AS make useful selectable markers for plant transformations. These markers work in conjunction with tryptophan analogs to identify transformants. ASA2 is a preferred selectable marker.

Tryptophan appears routinely in plant media as a component of casein hydrolysate. The presence of minute concentrations of tryptophan in selection media can allow non-transformants to escape the selection process, thereby yielding false positives.

It was discovered that selecting transformants on media substantially free of tryptophan enhanced the selection process by reducing the number of false positives. Thus, one aspect of the present invention provides a method of selecting a transformed plant cell comprising transforming a plant cell with a vector comprising a gene of interest and a gene encoding a feedback-insensitive form of anthranilate synthase (AS), and growing the transformed cell on a media composition comprising a tryptophan analog, wherein the media is substantially free of tryptophan.

The methods, media and plasmids herein described are useful in selection of transgenic forest tree plants or lines using selection agents that alter amino acid metabolism, such as sulfonylurea and imidazolinone herbicides and methylated tryptophan analogs. They are further useful in selection, pre-selection and pre-transformation media used to culture plant material to be subjected to transformation with selectable markers that alter amino acid metabolism.

The methods, media and plasmids described above are further useful in the regeneration of plants from cell lines that have been selected using selection agents that alter amino acid metabolism. They can be for obtaining plants transformed with selectable markers that alter amino acid metabolism and that also may be resistant to herbicides that alter amino acid metabolism. Accordingly, the inventions herein also are useful for obtaining herbicide-resistant treestocks. In addition, the inventions are useful in providing crop material suitable for herbicide management of weeds in forestry plantings.

The inventive methods, media and plasmids can be used in the selection and regeneration of plants transformed with genes that result in overproduction of specific amino acids such as tryptophan. Accordingly, they may be further useful for obtaining treestocks showing increased or altered growth as a result of such overproduction.

The methods described herein are generally useful for developing and testing new selection media and selective agent doses. The same principles used to design these media can be applied to the design of selection media for positive selection methods, such as the use of normally non-metabolized sugars.

The inventive methods also are useful for developing formulations of amino acid mixtures to supplement plant tissue culture media.

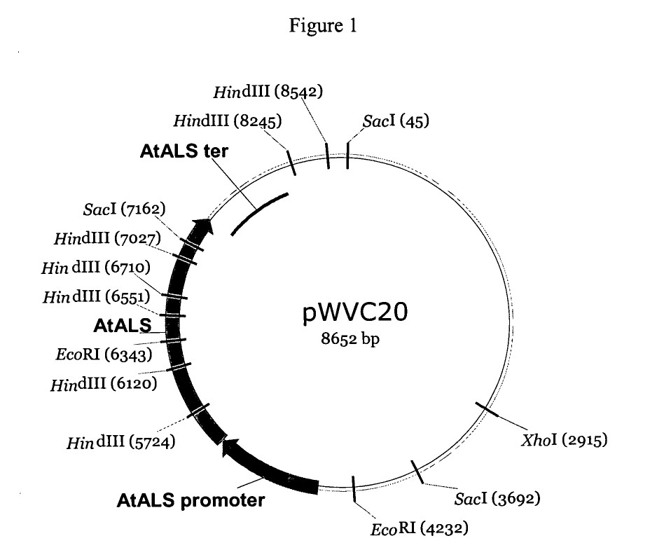

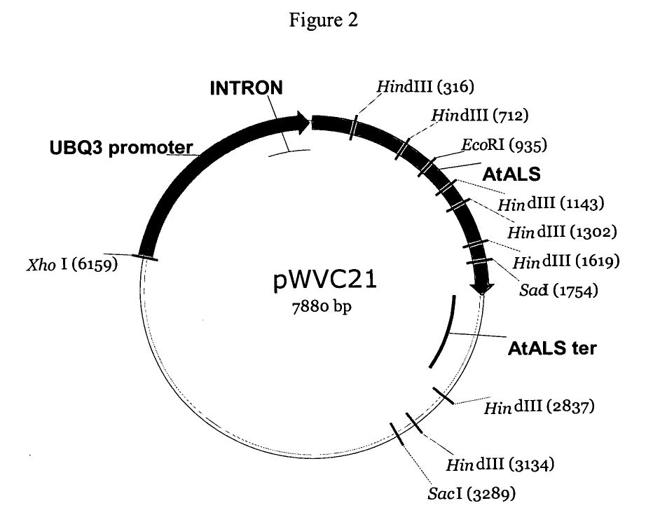

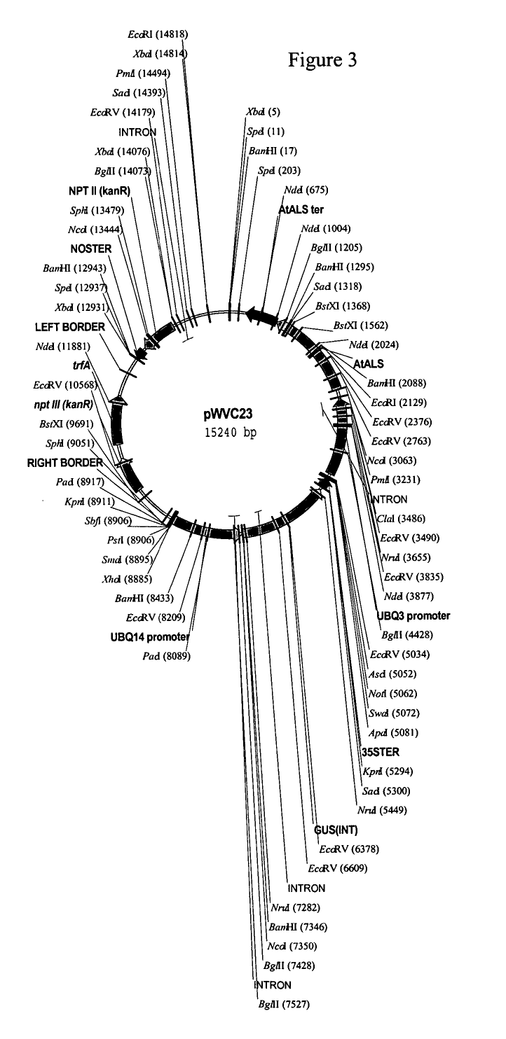

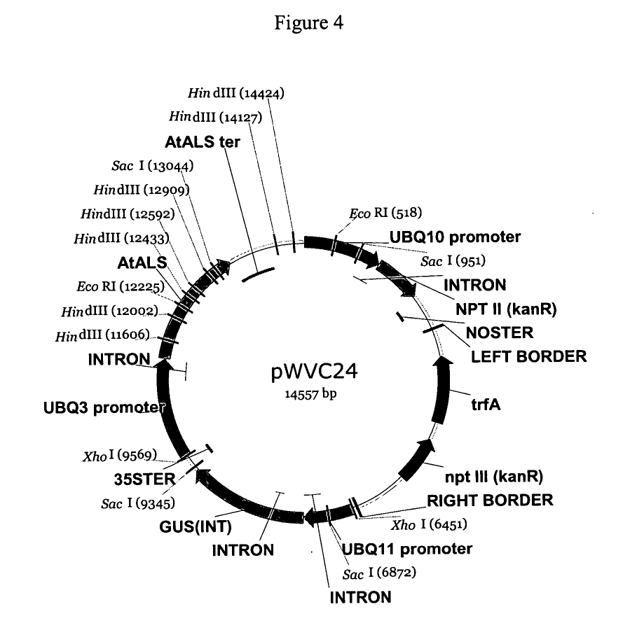

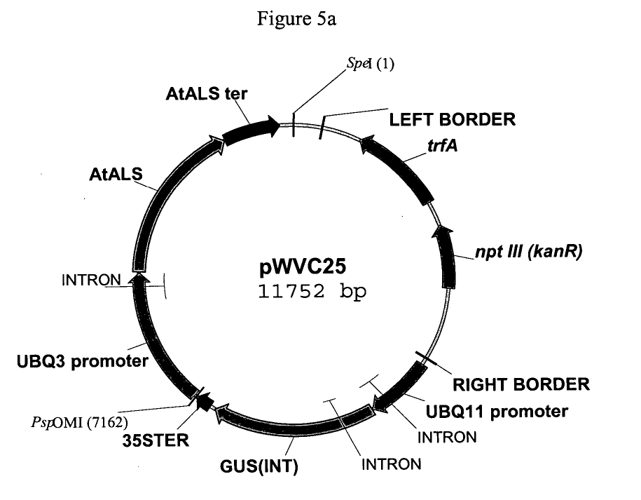

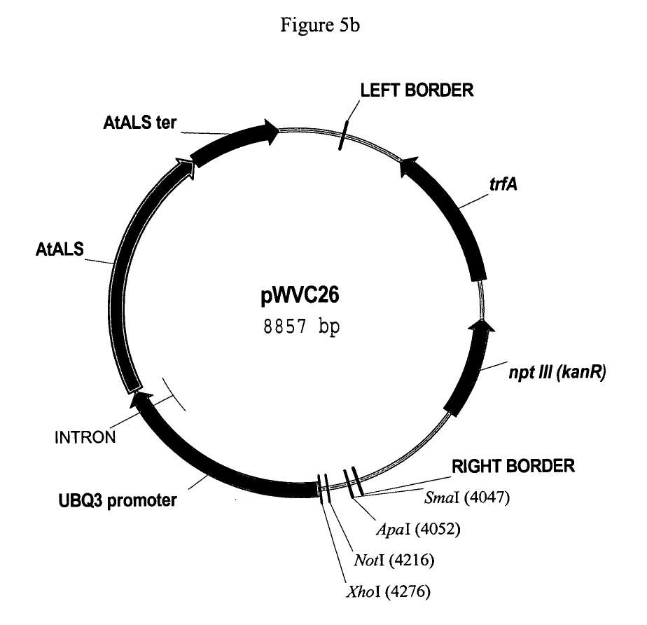

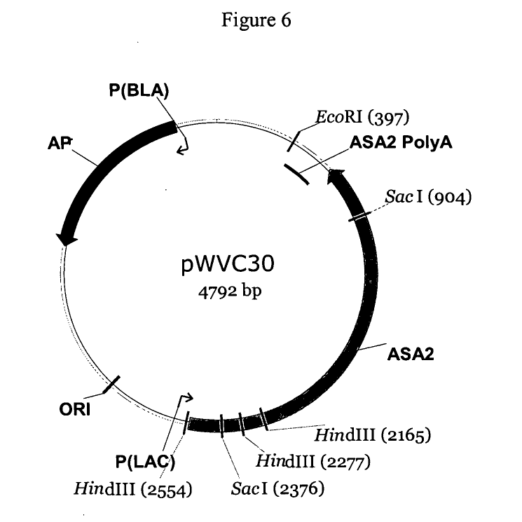

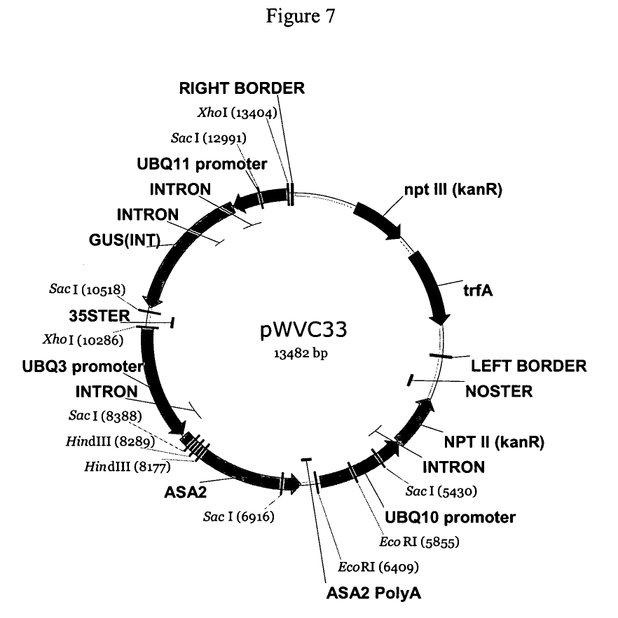

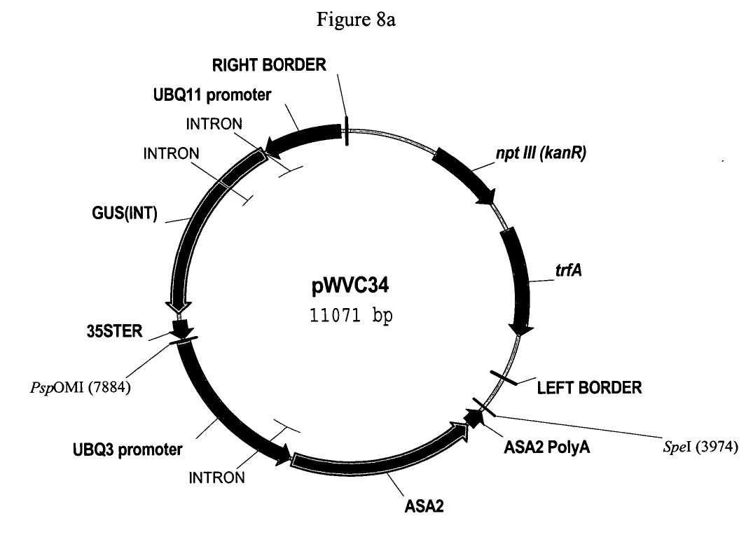

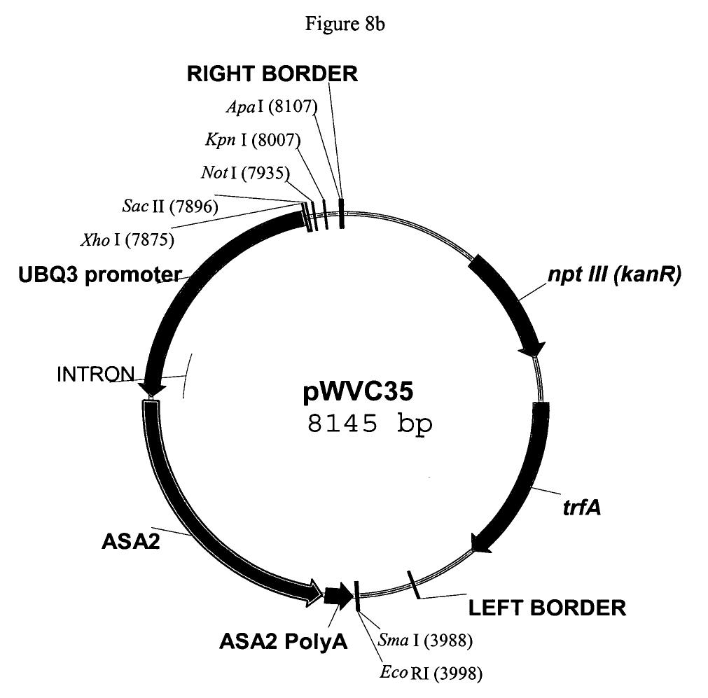

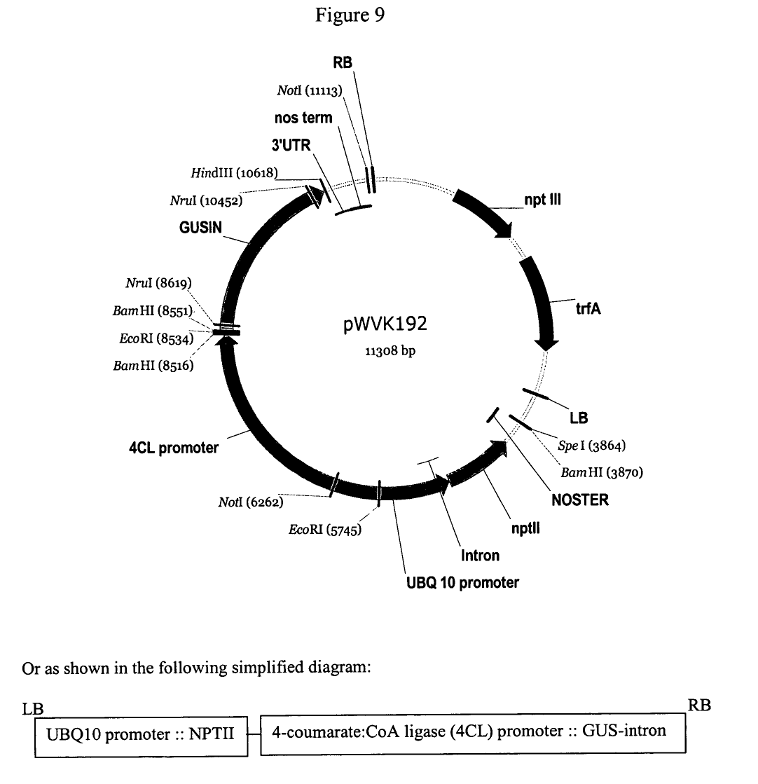

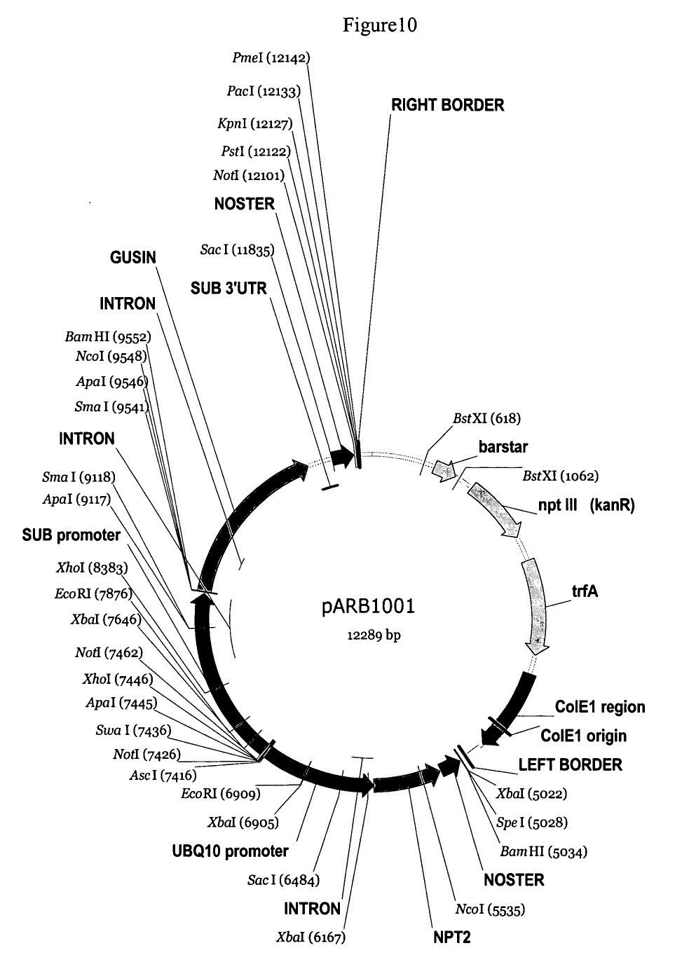

In another embodiment, the invention provides recombinant constructs useful for expressing heterologous proteins in plants. Examples of the inventive vectors include the following: pWVC20 (FIG. 1), pWVC21 (FIG. 2), pWVC23 (FIG. 3), pWVC24 (FIG. 4), pWVC25 (FIG. 5a), pWVC26 (FIG. 5b), pWVC30 (FIG. 6), pWVC33 (FIG. 7), pWVC34 (FIG. 8a), pWVC35 (FIG. 8b), and pWVK192 (FIG. 9), and pARB1001 (FIG. 10).

Another aspect of the invention provides methods of obtaining wood, wood pulp, paper, and oil from a plant transformed and selected by the methods of the present invention. Methods for transforming and selecting a transgenic plant are provided above and are known in the art. A transformed plant can be cultured or grown under any suitable conditions. For example, pine can be cultured and grown as described in U.S. Patent Application Publication No. 2002/0100083. Eucalyptus can be cultured and grown as in, for example, Rydelius, et al., Growing Eucalyptus for Pulp and Energy, presented at the Mechanization in Short Rotation, Intensive Culture Forestry Conference, Mobile, Ala., 1994. Wood, wood pulp, paper, and oil can be obtained from the plant by any means known in the art.

As noted above, the wood and wood pulp obtained in accordance with this invention may demonstrate improved characteristics including, but not limited to any one or more of lignin composition, lignin structure, wood composition, cellulose polymerization, fiber dimensions, ratio of fibers to other plant components, plant cell division, plant cell development, number of cells per unit area, cell size, cell shape, cell wall composition, rate of wood formation, aesthetic appearance of wood, formation of stem defects, rate of growth, rate of root formation ratio of root to branch vegetative development, leaf area index, and leaf shape include increased or decreased lignin content, increased accessibility of lignin to chemical treatments, improved reactivity of lignin, increased or decreased cellulose content increased dimensional stability, increased tensile strength, increased shear strength, increased compression strength, increased shock resistance, increased stiffness, increased or decreased hardness, decreased spirality, decreased shrinkage, and differences in weight, density, and specific gravity.

Phenotype can be assessed by any suitable means. The plants can be evaluated based on their general morphology. Transgenic plants can be observed with the naked eye, can be weighed and their height measured. The plant can be examined by isolating individual layers of plant tissue, namely phloem and cambium, which is further sectioned into meristematic cells, early expansion, late expansion, secondary wall formation, and late cell maturation. See, e.g., Hertzberg, supra. The plants also can be assessed using microscopic analysis or chemical analysis.

Microscopic analysis includes examining cell types, stage of development, and stain uptake by tissues and cells. Fiber morphology, such as fiber wall thickness and microfibril angle of wood pulp fibers can be observed using, for example, microscopic transmission ellipsometry. See Ye and Sundström, Tappi J., 80:181 (1997). Wood strength, density, and grain slope in wet wood and standing trees can be determined by measuring the visible and near infrared spectral data in conjunction with multivariate analysis. See, U.S. Patent Application Publication Nos. 2002/0107644 and 2002/0113212. Lumen size can be measured using scanning electron microscopy. Lignin structure and chemical properties can be observed using nuclear magnetic resonance spectroscopy as described in Marita et al., J. Chem. Soc., Perkin Trans. 12939 (2001).

The biochemical characteristic of lignin, cellulose, carbohydrates and other plant extracts can be evaluated by any standard analytical method known including spectrophotometry, fluorescence spectroscopy, HPLC, mass spectroscopy, and tissue staining methods.

It will be readily apparent to one of ordinary skill in the relevant arts that other suitable modifications and adaptations to the methods and applications described herein can be made without departing from the scope of the invention or any embodiment thereof.

The following examples are set forth as representative of specific and preferred embodiments of the present invention. These examples are not to be construed as limiting the scope of the invention in any manner. It should be understood that many variations and modifications can be made while remaining within the spirit and scope of the invention.

### EXAMPLES

### Example 1

### Plant Materials

All elite clones were maintained as shoot clumps in Magenta boxes and subcultured every 4-6 weeks. Shoot clumps were divided as needed and maintained as stock culture. Unless noted otherwise, all cultures were grown under shaded, cool fluorescent light with a light intensity of 30-40 μE/m²/s with a photoperiod of 16 hours, and the temperature of the growth room is 21° C. Except for the selected E. dunnii clones, leaf explants were used for all selected clones.

Commercial E. dunnii elite clone DUN00001 were provided by Rigesa, a subsidiary of MeadWestvaco Brazil. Shoot clumps were maintained at 4 clumps per Magenta box. The preferred explant for these clones are tissues with advanced vascular tissues, such as internodes, petioles, and midribs.

E. grandis clones were provided by Rubicon, New Zealand. E. grandis clones were received as shoot clumps on solid medium. The clumps were transferred to Euc Maintenance medium (Table 1) with BA at 0.25-0.5 mg/l in Magenta boxes. The shoot clumps were transferred to fresh medium every 4-6 weeks.

Commercial E. urophylla clones IPB1 and IPB10, E. grandis×urophylla hybrid clones IPB3, IPB6, 530 and 736, and E. saligna clone IPB13 were provided by International Paper, USA. Shoot clumps were received the same way as described for E. grandis. The stock establishment procedures and the culture conditions were the same as with E. grandis, except that all cultures were grown under full light conditions at an intensity of about 120 μE/m²/s.

Commercial E. camaldulensis clones were received as cuttings from International Paper, USA. Newly flushed apical and axillary shoots were surface sterilized with 10-15% commercial bleach for 5-10 minutes, a quick rinse with 70% ethanol, and three rinses with sterile water. Nodes were separated and cultured on Euc Maintenance medium. When axillary shoots initiate from the nodes, they were transferred to fresh Euc maintenance medium for further proliferation. Stock cultures were established on the same medium after 3-4 culture cycles. The stock cultures were maintained under the same condition as previously described for the other clones.

Early flowering E. occidentalis clones were maintained in the greenhouse. Newly flushed apical and axillary shoots were surface sterilized with 10-15% commercial bleach for 5-10 minutes, a quick rinse with 70% ethanol, and three rinses with sterile water. Nodes were separated and cultured on Euc Maintenance medium. When axillary shoots were initiated from the nodes, they were transferred to fresh Euc maintenance medium for further proliferation. Stock cultures were established on the same medium after 3-4 culture cycles. The stock cultures were maintained under the same condition as previously described for the other clones.

### Example 2

### Construct

Six constructs were used for the transformation experiments: pWVZ20, pWVR133, pWVR31, pARB1001, pWVK192, and p35SGUSIN. Binary plasmid pWVZ20 contains an herbicide resistant gene driven by a plant promoter and the β-glucuronidase (GUS) gene driven by a constitutive promoter between T-DNA borders. Binary plasmid pWVR133 contains the same herbicide resistant gene construct, but the GUS gene contains an intron and is driven by an actin promoter. GUS expression of pWVR133 only occurs in plant cells but not in bacterial cells. Binary plasmid pWVR31 has the intron-containing GUS gene driven by ubiquitin 11 promoter and the neomycin phosphotransferase II gene (NPTII) under the ubiquitin 10 promoter. pWVK192 consists of the same selectable marker gene as in pWVR31, but the intron-containing GUS gene is driven by a promoter that expresses in the regeneration stage as well as in the xylem of well developed plants. p35SGUSIN consists of the same marker genes as in pWVR31 except that the GUS gene is driven by 35S promoter and the NPTII gene is driven by nopaline synthase promoter (NOS promoter).

Binary plasmid pARB1001 was constructed from a binary vector derived from pBIN19 reduced in size through deletion of nonessential DNA segments, pARB310, to which a gene for barstar (Hartley, 1988) that had been previously cloned with flanking BstXI sites was removed from pWVR200B by BstXI digestion and gel purified. The approximately 470 bp fragment was ligated into pARB310 that had been digested with BstXI to make pARB310B. Next, the ColE1 replication origin and surrounding region were amplified from pART27 (Gleave, 1992) using PCR with the primer pair, ColE1-F4 (5′-GAGAGAGGATCCGGTGTGAAATACCGCACAG-3′) and ColE1-R4 (5′-GAGAGATGATCAGCCTCACTGATTAAGCATTGGTAACTG-3′). The 1.0 kb ColE1 fragment was digested with BamHI and BclI, then purified and ligated into the BclI site of pARB310B, between the end of the trfA gene and the left border (LB) of the T-DNA. This generated pAGF50, which provided a high copy number plasmid in E. coli, but still replicated in Agrobacterium. pAGF50 was digested with AscI and NcoI to remove the UBQ3 promoter plus most of the NPTII coding region, and the resulting 5.7 kb fragment was gel purified. The 1.9 kb fragment with UBQ10 promoter linked to the 5′-end of the NPTII coding region was released from pWVR3 by AscI and NcoI digestion, gel purified, and ligated into the pAGF50 fragment to generate pARB1000. This plasmid was further modified by the addition of a SUBIN::GUSIN::NOSTER reporter cassette. SUBIN indicates a ubiquitin promoter from P. radiata, SEQ ID NO: 2 of U.S. Pat. No. 6,380,459, which included genomic DNA coding the 5′-UTR and an intron; GUSIN indicates the β-glucuronidase coding region plus an intron from the potato tuberin gene (Vancanneyt et al., 1990). The reporter cassette was removed from pARB494 by DraI digestion and ligated into the SmaI site of pARB1000 to generate pARB1001.

The Agrobacterium strain GV2260 (Deblaere et al. 1985) was used for the transformation studies. One of ordinary skill in the art would be able to use any binary construct having an herbicide resistant gene operably linked to a constitutive plant promoter.

### Example 3

### Preparation of Agrobacterium for Transformation

Agrobacterium containing either pWVZ20, pWVR133, pWVR31, pWVK192, pARB1001, or p35SGUSIN were grown on YEP medium (10 g/l yeast extract, 10 g/l peptone and 50 mg/l NaCl, pH 7.0-7.2) for 3 days. A single colony from the plate was selected and grown in 20-50 ml liquid YEP medium with kanamycin at 100 mg/l and rifampicin 50 mg/l. Cultures were incubated overnight in a shaking incubator at 28° C. and 150 rpm. The over night culture with an OD of about 0.6-1.1 was spun down in a desktop centrifuge at 3000 g for 20 minutes and resuspended with Agrobacterium Induction Medium or AIM (WPM salts with glucose at 5 g/l, 250 μM acetosyringone, 2 mM phosphate buffer, and 0.05 M MES, pH 5.8). In experiments with pARB1001 and clones 530 and 736, a lower optical density of 0.4 was targeted. Cultures were incubated for 25 minutes at 28° C. before infection. Bacterial concentration was determined before and after infection.

### Example 4

### Preparation of Explants for Infection and Pre-Culture

Eucalyptus stock cultures maintained on Euc Maintenance medium were used as the sources of the explants. Although leaves, petioles, internodes, floral tissues, and embryogenic tissues can be used for transformation, leaf, petiole and internode explants were used in the following examples. Leaf explants were the most commonly used explants because of their abundance. Petioles or internodal explants were from healthy rooted plants. Healthy and newly opened leaves were from shoot clumps on maintenance medium. The tip portions of the leaves were removed by scissors or forceps to increase the number of wounded cells. Explants were placed on pre-culture medium (Table 2), and the abaxial side is down for leaf explants. The pre-culture medium is a nutrient medium upon which plant explants are cultured before Agrobacterium transformation. Specifically, the inventive pre-culture medium increases transformation efficiency and plant regeneration. While the present pre-culture medium comprises acetosyringone, other Agrobacterium inducers may be used. Pre-culture medium in Table 2 is the preferred pre-culture medium; however, other salt media, such as MS medium (Murashige and Skoog, 1962), modified Woody Plant Medium (WPM), or Lepoivre medium, may be used. In the present invention, plant explants were pre-cultured for either one or four days in the dark on the pre-culture medium displayed in Table 2. While not required, the instant pre-culture medium contained elevated auxin levels. Additionally, plant explants may be cultured on the pre-culture medium from one to six days before Agrobacterium transformation.

For experiments with E. grandis×E. urophylla hybrid clones 530 and 736 using pARB 1001, simultaneous explanting was done according to the method disclosed in PCT Application Publication No. WO 00/12715 for comparison. The explants collected according to the latter method were pre-cultured for one day on the pre-culture medium.

### Example 5

### Agrobacterium Inoculation and Removal

Induced Agrobacterium culture was prepared as described in Example 3 and the culture was dripped onto each explant by pipette. Sufficient Agrobacterium culture was dripped to ensure that all the cut edges were covered with bacterial solution. Alternatively, the explants may be transformed by vacuum infiltration, floral dip, and other methods of Agrobacterium-mediated transformation. Following transformation, explants covered with Agrobacterium culture were placed in the dark for three or four days of co-cultivation. Alternatively, the explants may be co-cultivated with Agrobacterium under light conditions. Additionally, the explants may be co-cultivated with Agrobacterium under light or dark conditions for 2-10 days, preferably 3 days. Following co-cultivation, the explants were transferred to Euc Regeneration medium (Table 3) with 400 mg/l timentin. There is no need to wash explants. Explants were cultured on this medium for four days before transfer to a selection medium. In the present example, the selection medium is the Euc Regeneration medium supplemented with both timentin and an herbicide selection agent.

For experiments with E. grandis×E. urophylla hybrid clones 530 and 736, simultaneous infection was done according to the method of PCT Application Publication No. WO 00/12715 for comparison, and the explants were co-cultured following infection for three days in the dark. However, infection using the method disclosed in PCT Application Publication No. WO 00/12715 resulted in less than 20% responsive explants, and therefore the inventive method described in this patent application is preferred.

### Example 6

### Regeneration of Transgenic Shoots

Shoot clumps that survive selection are maintained on Euc regeneration medium containing either herbicide or antibiotic for selection and timentin for Agrobacterium control, and they are transferred every 3 weeks until shoots proliferate and initially elongate. For transformation experiments with pWVR133 and pWVK192, leaf and stem tissues from the regenerated shoots are stained for GUS expression as soon as the shoots are developed. For transformation experiments with pWVZ20, leaf and stem tissues from the regenerated shoots are stained for GUS expression when the shoots are further developed and free from residual Agrobacterium.

For the transformation and regeneration of E. grandis×E. urophylla clone 530, the pre-culture/co-cultivation medium omitted the casein hydrolysate and glutamine, and both the pre-culture/co-cultivation medium and the regeneration medium were supplemented with an additional 0.375 mg/L MgSO₄. Table 5 bleow shows E. grandis×E. urophylla clone 530 shoots transformed with an intron-containing GUS gene. Notably, the stable GUS expression in E. grandis×E. urophylla clone 530 indicates that the intron is correctly excised and the gene product is functional.

TABLE 5
Identification of E. grandis x E. urophylla clone 530 transgenic
shoots using constructs with intron-containing GUS gene.

| | Geneticin | | | |
|---|---|---|---|---|
| No. | Selection | No. | No. Shoots | No. GUS+ |
| Explants | (mg/l) | Shoots/Primordia | Stained | Shoots |
| 141 | 40 | 4 | 2 | 2 |
| 144 | 50 | 4 | 2 | 2 |

### Example 7

### GUS Staining

GUS staining was performed to monitor the frequency of Agrobacterium infection and to ensure that the selected shoots are not escapes or chimeras. For transformation experiments with pARB1001, cultures were stained four days after transformation. For transformation experiments with pWVR133, pWVR31, pWVK192, and p35SGUSIN the leaf and stem tissues from the regenerated shoots were stained for GUS expression immediately upon shoot development. For transformation experiments with pWVZ20, leaf and stem tissues from the regenerated shoots were stained for GUS expression when shoots are further developed and free from residual Agrobacterium. To determine GUS activity, the explants were incubated in a substrate comprising 100 mM phosphate buffer (pH 7.0), 0.05% dimethyl suphoxide, 0.05% Triton X-100, 10 mM EDTA, 0.5 mM potassium ferrocyanide, and 1.5 mg/ml 5-bromo-4-chloro-3-indolyl-β-D-glucuronide (X-gluc). The explants were subjected to 10 minutes of vacuum before an overnight incubation at 37° C. Following overnight incubation, GUS foci were counted. As shown below in Table 6, the inventive method provides a higher infection rate than the method of WO 00/12715.

TABLE 6
Auxin Pre-treatment Increases Agrobacterium Infection Rate
for E. grandis × E. urophylla clone.

| | | | | | | Average | Number |
|---|---|---|---|---|---|---|---|
| | | | # | % | Total | number of | of foci |
| E. grandis × | | | Explants | Expalnts | number | blue foci | rated as |
| E. urophylla | Infection | # | w/blue | w/blue | of blue | per | heavily |
| clone | Method | Explants | foci | foci | foci | explant | blue |
| 530 | WO | 20 | 3 | 15 | 5 | 1.67 | 0 |
| | 00/12715 | | | | | | |
| 736 | WO | 20 | 4 | 20 | 5 | 1.25 | 0 |
| | 00/12715 | | | | | | |
| 530 | Present | 21 | 20 | 95 | 111 | 5.55 | 10 |
| | method + 1 | | | | | | |
| | day | | | | | | |
| | preculture | | | | | | |
| 736 | Present | 21 | 19 | 95 | 195 | 10.26 | 3 |
| | method + 1 | | | | | | |
| | day | | | | | | |
| | preculture | | | | | | |

In conclusion, the present method was preferred over the method of PCT publication No. WO 00/12715 because the latter method resulted in a much smaller percentage of responsive explants for E. grandis×E. urophylla.

### Example 8

### Regeneration of Shoots in Media Containing Inventive Amino Acid Mixture

This example teaches the use of an amino acid and vitamin mix (see Table 3) to accelerate the shoot regeneration process by promoting the growth of shoot masses. Leaf explants of E. grandis×E. urophylla clones 530 and 736 were prepared as described in Example 1 and Agrobacterium inoculum was prepared according to Example 4. The leaf explants were inoculated as described in Example 5 and co-cultivated for three days. Following co-cultivation, leaf explants were harvested and placed on medium containing the amino acid mix at 10 pieces per Magenta box. At 4 weeks, the early phase of organogenesis was evaluated as the frequency of explants forming adventitious shoots and the size of the clumps with shoot primordia, small shoots and callus was measured. As displayed in Table 7, the inventive regeneration media promotes E. grandis×E. urophylla shoot development.

TABLE 7
Regeneration of E. grandis x E. urophylla explants
after 33 days of culture.

| | | No. explants | No. explants |
|---|---|---|---|
| | No. explants with callus | with very young | with well |
| Clone | only | shoot primordia | developed shoots |
| 736 | 40 | 8 | 2 |
| 530 | 18 | 0 | 1 |

This example shows that the present media are able to support regeneration of shoots from E. grandis×E. urophylla clones.

### Example 9

### Explant Tissue Type Varies Infection

This example teaches a method to screen for a preferred explant for transformation. Leaf, petiole and stem explants were isolated from elite E. grandis×urophylla clone IPB3 plants growing on WPM with 0.5 mg/l BA. Leaf explants were taken from young apical or axillary shoots, excised along the midribs into rectangular pieces with length and width of around 4-6 mm. Petioles or internodes were from the top half of shoots and cut into 4-6 mm sections. Agrobacterium strain GV2260 containing pWVR133 was prepared as described in Example 3. Harvested explants were incubated in the dark for 4 days on Eucalyptus regeneration medium. Agrobacterium culture was dripped on explants and co-cultured with the explants on the same medium for 3 days in dark. Then, explants were removed from the Agrobacterium puddle and transferred to Euc Regeneration medium supplemented with 400 mg/l timentin for 4 days. Following transfer to regeneration medium, the explants were stained for GUS expression. As shown in Table 8, leaf and petiole explants were more amenable to infection than internode explants.

TABLE 8
Leaf and Petiole Explants Have Increased Infection Rates Than Stem
Explants For an Elite Eucalyptus grandis x urophylla Clone IPB3

| Explant | No. Explants | No. GUS+ | % GUS+ |
|---|---|---|---|
| Leaf | 42 | 6 | 14.3 |
| Petiole | 7 | 6 | 14.3 |
| Stems | 62 | 16 | 1.6 |

### Example 10

### Agrobacterium strains, GV2260 and EHA105, and Culture Vessels on Transformation of Elite Eucalyptus grandis×urophylla clone IPB6

This example compares the infection of either leaf or internodal explants with two Agrobacterium strains, GV2260 and EHA105. The pre-culture and co-cultivation medium are the regeneration medium with the combination of 250 μM AS and an elevated amount of auxin as IAA at 2 mg/l IAA (vs. 0.1 mg/l NAA in regular regeneration medium). The pre-culture and co-cultivation medium are the regeneration medium with the combination of 250 μM AS and an elevated amount of auxin as IAA at 2 mg/l IAA. The plasmid used in this experiment was pWVR31. Leaf explants of about 1-2 mm wide were used for the pre-culture, co-cultivation and recovery as described in previous examples. As shown in Table 9 below, the explants cultured in boxes undergo a similar frequency of infection as the explants cultured in plates, but infection in boxes can result in higher number of GUS foci when using EHA105.

TABLE 9
E. grandis × urophylla Clone IPB6 Has Similar Infection
Frequency with Agrobacterium strains EHA and GV2260

| | | | | | | Avg. No. |
|---|---|---|---|---|---|---|
| | | No. | | | No. | Foci/ |
| Agrobacterium | | Ex- | No. | % | GUS | Responding |
| strain | Vessels | plants | GUS+ | GUS+ | Foci | Explant |
| EHA | Boxes | 100 | 31 | 31 | 82 | 2.6 |
| EHA | Plates | 100 | 33 | 33 | 57 | 1.7 |
| GV2260 | Boxes | 97 | 23 | 24 | 42 | 1.8 |

### Example 11

### RNA Isolation

RNA was isolated from leaf and shoot tissues of wild-type and transformed E. grandis×E. urophylla with RNAqueous™-96 kit, according to the manufacturer's instructions. Briefly, 0.1 to 1.5 mg tissue was suspended in 300 μl Lysis/Binding buffer and ground to a fine powder with a plastic pestle. The samples were centrifuged at maximum speed for 3 minutes and the supernatant was transferred to a fresh plate. Three hundred μl (1x v/v) 64% ethanol was added to the supernatant and the samples were vortexed briefly. The samples were then centrifuged for 2 minutes at 1850 g. Following centrifugation, an optional DNase treatment step was performed. To each sample, 5 μl DNase+35 μl DNase buffer was added to the center of the filter pad and the samples were incubated at room temperature for 20 minutes. After the 20 minute incubation, 600 ml Wash Buffer A was added to each sample and the samples were centrifuged at maximum speed for 2 minutes. Following centrifugation, the supernatants were discarded and 600 μl Wash Buffer B was added to the filter pad of each sample. Following centrifugation at maximum speed for 2 minutes, the supernatants were discarded and 600 μl Wash Buffer C/D was added to each filter pad. The samples were centrifuged at maximum speed for 2 minutes and the supernatants were discarded. Following a second rinse with Wash Buffer C/D, 50 μl RNA elution solution was added and the samples were centrifuged at maximum speed for 2-3 minutes. The RNA elution step was repeated and the eluted RNA samples were stored at −80 degrees Celsius until further use.

### Example 12

### RT-PCR

To determine the presence and expression of the ALS herbicide resistance gene, RT-PCR was performed. Total RNA was isolated, as described in Example 12, from transformed and wild-type samples of E. grandis×E. urophylla plants. Following RNA isolation and quantitation, RT-PCR was performed with Ready to Go RT-PCR kit (Pharmacia), according to the manufacturer's instructions. Generally, 5 μl total RNA (about 20 ng RNA) was added to a 45 μl RT-PCR reaction mixture containing 1 μl Oligo dTn, 1 μl 3 μM ALS forward primer, 1 μl 3 μM ALS reverse primer, and 42 μl water.

To denature the RNA, the tubes were heated to a temperature of at least 75° C. for 3 minutes. The samples were then incubated at 42° C. for 30 minutes.

Following incubation, RT-PCR was performed as follows:

| | |
|---|---|
| Cycle 1: | 95° C. for 5 minutes |
| | 55° C. for 1 minute |
| | 72° C. for 1 minute |
| Cycles 2-30 | 95° C. for 1 minute |
| | 55° C. for 1 minute |
| | 72° C. for 1 minute |
| Cycle 31 | 95° C. for 1 minute |
| | 55° C. for 1 minute |
| | 72° C. for 10 minutes |

Following completion of RT-PCR, the PCR amplification products were visualized on an agarose gel stained with ethidium bromide.

### Example 13

### RT-PCR to Assess Expression of Herbicide Tolerance Gene

This example shows the presence and expression of a herbicide tolerance gene in a sample of transgenic lines. As described in Example 11, RNA was isolated from the leaves and stem tissues of each putative transgenic line. In particular, RNA was isolated from transformed E. grandis, E. grandis×urophylla, and E. camadulensis lines. In addition, RNA was isolated from wild-type E. grandis, E. grandis×urophylla, and E. camadulensis plants. For each line, a 5 μl RNA sample was used for RT-PCR to assess the presence and the expression of a herbicide tolerance gene. RT-PCR was performed with the Ready-to-Go RT-PCR kit (Pharmacia), according to the manufacturer's instructions. Briefly, each 5 μl RNA preparation was added to separate RT-PCR reactions containing oligo(dT)ₙ and herbicide-resistant gene primers. Following RT-PCR, the amplification products were visualized on an ethidium bromide-stained agarose gel.

Agarose gel electrophoresis of the RT-PCR products revealed that all of the tested transgenic Eucalyptus lines express the herbicide tolerance gene. Specifically, all eight of the E. grandis×urophylla express, while the control plants do not express ALS. No herbicide tolerance gene expression was detected in any of the control plants.

What is claimed is:

1. A method for transforming at least one cell of a E. grandis×E. urophylla explant with a foreign DNA, comprising
(i) culturing a tree explant on a pre-culture medium comprising an inducer of Agrobacterium;
(ii) exposing said explant to an Agrobacterium strain containing a transformation vector carrying said foreign DNA;
(iii) selecting a transformed explant, wherein said foreign DNA is transferred to at least one cell of said transformed explant; and
(iv) regenerating said transformed explant to produce a complete plant.

2. The method of claim 1, wherein said inducer of Agrobacterium is acetosyringone.

3. The method of claim 2, wherein the concentration of said acetosyringone is from about 10 to about 400 mg/l.

4. The method of claim 3, wherein the concentration of said acetosyringone is from about 5 to about 200 mg/l.

5. The method of claim 1, wherein said medium further comprises auxin.

6. The method of claim 1, wherein said medium further comprises cytokinin.

7. The method of claim 1, wherein said explant is pre-cultured in the dark from about 1 to about 6 days.

8. The method of claim 1, wherein said explant is pre-cultured for about 4 days.

9. The method of claim 5, wherein said auxin is selected from the group consisting of NAA, 2,4-D, IBA, and IAA.

10. The method of claim 9, wherein the concentration range of any one of NAA, 2,4-D, IBA, and IAA is from about 0.1 to about 10 mg/l.

11. The method of claim 10, wherein said concentration range is from about 0.2 to about 5 mg/l.

12. The method of claim 11, wherein said concentration range is from about 0.2 to about 3 mg/l.

13. The method of claim 6, wherein said cytokinin is selected from the group consisting of zeatin, kinetin, and BA.

14. The method of claim 13, wherein the concentration range of any one of said zeatin, kinetin, and BA is from about 0.25 to about 15 mg/l.

15. The method of claim 14, wherein said concentration range is from about 1 to about 10 mg/l.

16. The method of claim 15, wherein said concentration range is from about 1 to about 6 mg/l.

17. The method of claim 1, wherein said explant is at least one of a leaf, a petiole, an internodal tissue, a floral tissue, or an embryogenic tissue.

18. The method of claim 1, wherein said tissues are selected independent of age or developmental stage.

19. The method of claim 1, wherein said method is genotype-independent.

20. The method of claim 1, wherein all of the cells of said transformed explant comprise said foreign DNA.

21. A method for producing a non-chimaeric E. grandis×E. urophylla tree, comprising
(i) pre-culturing a E. grandis×E. urophylla explant on a medium comprising an inducer of Agrobacterium;
(ii) exposing said explant to an Agrobacterium strain harboring a vector capable of transferring a gene to a plant cell;
(iii) selecting a transformed explant; and
(iv) growing said explant to produce a non-chimaeric tree.


SEQUENCE LISTING (2 sequences)

SEQ ID NO: 1 - DNA, 31 bp, Artificial Sequence
  Description of Artificial Sequence Synthetic primer
gagagaggatccggtgtgaaataccgcacag

SEQ ID NO: 2 - DNA, 39 bp, Artificial Sequence
  Description of Artificial Sequence Synthetic primer
gagagatgatcagcctcactgattaagcattggtaactg